(19) United States
(12) Patent Application Publication
(10) Pub. No.: US 2026/0199541 A1
(43) Pub. Date: Jul. 16, 2026

(54) AIR DISINFECTION AND BIOSECURITY SYSTEMS

(71) Applicant: PATHOGENFOCUS LLC, Henderson, NC (US)

(72) Inventors: Charles E. Kassay, New Bern, NC (US); Marc A. Kassay, Wake Forest, NC (US)

(21) Appl. No.: 19/136,131

(22) PCT Filed: Dec. 20, 2023
(86) PCT No.: PCT/US23/85128

§ 371 Date: Jun. 5, 2025

Related U.S. Application Data
(60) Provisional application No. 63/434,720, filed on Dec. 22, 2022.

(51) Int. Cl. A61L 9/015; A61L 9/22; C01B 15/027; F24F 8/26

(52) U.S. Cl. CPC A61L 9/015; A61L 9/22; C01B 15/027; F24F 8/26; A61L 2209/111; A61L 2209/134; A61L 2209/14; A61L 2209/16; A61L 2209/211; A61L 2209/212

(57) ABSTRACT

An fluid disinfection biosecurity (ADB) system comprising a housing with an inlet and outlet, and an conduit positioned between the inlet and outlet, the conduit configured to direct air from the inlet to the outlet. The ADB system also includes a reaction chamber positioned in the conduit between the inlet and the outlet; a high frequency module and sequential relay module set configured to deliver an electrical pulse at a frequency and intensity; and a distribution board coupled to the high frequency module and sequential relay module set. The distribution board may be configured to control the frequency and intensity of the electrical pulse by delivering voltage to the high frequency module and sequential relay module set. The high frequency module and sequential relay module set interacts with the reaction chamber to generate cold plasma which is delivered from the ADB system via air.

### RELATED APPLICATIONS

This application is a U.S. national stage application under 35 U.S.C. §371 of PCT International Application Ser. No. PCT/US2023/085128, filed Dec. 20, 2023, which claims priority to U.S. Provisional Patent Application Ser. No. 63/434,720, filed Dec. 22, 2022, the entire contents of which are incorporated herein by reference in their entirety.

### FIELD OF THE DISCLOSURE

The present disclosure relates to air and surface disinfection systems, and more particularly to air and surface disinfection systems utilizing air disinfection and biosecurity technology.

### BACKGROUND

Due to the recent pandemic, it has become obvious to the vast majority of individuals that the eradication of pathogens, virus, bacteria, mold and fungus is a persistent challenge. People want to live without fear of infections, they want to move about freely, at work, school, recreation, travel, and other venues without fear of infection. This requires the development and installation of devices that will reduce or eliminate pathogens, virus, bacteria, mold and fungus and allow the fear of infection to become less of a concern.

People that occupy a room will contaminate indoor air by exhaling carbon dioxide, sweating, coughing, etc. In addition, paint, carpet, upholstery, and other fixtures emit minute particles and vapors into the air. The typical way to address these issues is to bring outdoor air into the room. The amount of outdoor air brought into the room can be measured in air changes per hour (ACH). This extra ventilation adds to energy costs since the outdoor air needs to be conditioned in temperature and other aspects as it is brought into the room. Increasing ACH will also increase energy costs to condition the air.

It would be desirable to have a system that can effectively reduce or eliminate pathogens, virus, bacteria, mold and fungus in large or small areas where a group of people might gather, or in areas where pathogens, virus, bacteria, mold and fungus might linger from occupancy to occupancy.

### SUMMARY

The present disclosure may comprise one or more of the following features and combinations thereof.

According to one aspect of the present disclosure, a fluid disinfection biosecurity device may include a housing with an inlet and an outlet. The fluid disinfection biosecurity device may include a conduit positioned between the inlet and the outlet, the conduit configured to direct fluid from the inlet to the outlet. The fluid disinfection biosecurity device may also include a reaction chamber positioned in the conduit between the inlet and the outlet and a high frequency module and sequential relay module set (HFM/SRM set) configured to transmit an electrical pulse at a frequency and an intensity. The HFM/SRM set may be positioned within the housing and configured to transmit the electrical pulse to the reaction chamber. The fluid disinfection biosecurity device may further include a distribution board coupled to the HFM/SRM set, the distribution board configured to control the frequency and intensity of the electrical pulse by providing a voltage to the HFM/SRM set. The electrical pulse from the HFM/SRM set may be transmitted to the reaction chamber at a predefined intensity and frequency. The reaction chamber is configured to produce hydrogen peroxide via the interaction with the electrical pulse from the HFM/SRM set, and the hydrogen peroxide being mixed with the fluid in the conduit and being transferred out of the housing through the outlet.

In some examples, the housing may be coupled to one or more of an HVAC or air handling system. The fluid disinfection biosecurity device may further include a plurality of HFM/SRM sets positioned in the housing, wherein each HFM/SRM set of the plurality of HFM/SRM sets may operate independently of the other plurality of HFM SRM sets. In some examples, the reaction chamber is configured to receive the electrical pulse from the HFM/SRM set and produce a predetermined amount of gaseous hydrogen peroxide and ozone, wherein the gaseous hydrogen peroxide and ozone is produced free of any harmful or volatile organic compounds.

According to some examples, the fluid disinfection biosecurity device may further include a controller coupled to the distribution board, the controller configured to control the intensity or frequency of the electric pulse. The fluid disinfection biosecurity device may also include a mass airflow sensor coupled to the controller, and the mass airflow sensor may be positioned proximal to the inlet and configured to detect the mass flow rate of the fluid. The fluid disinfection biosecurity device may also include a fan coupled to the conduit, the fan being controllable by the controller and configured to move the fluid into the conduit and deliver the mixture of hydrogen peroxide and fluid to a space with a defined volume.

The fluid disinfection biosecurity device may be a standalone device separate from an HVAC or air handling system. The fluid disinfection biosecurity device may also include a plurality of HFM/SRM sets, wherein the plurality of HFM/SRM sets may be positioned in series or parallel with respect to one another within the housing.

According to another aspect of the present disclosure, an air disinfection biosecurity system may include a plurality of air disinfection biosecurity devices. Each air disinfection biosecurity device may include a housing with an inlet and an outlet, and a reaction chamber positioned within the housing between the inlet and outlet; a conduit configured to move fluid from the inlet through the reaction chamber to the outlet. Each air disinfection biosecurity device may include a high frequency module and sequential relay module set (HFM/SRM set) configured to deliver an electrical pulse to the reaction chamber to generate hydrogen peroxide, the electrical pulse having a predetermined intensity and frequency and a distribution board coupled to the HFM/SRM set configured to control the electrical pulse. Each air disinfection biosecurity device may also include a controller communicatively coupled to one or more of the plurality of air disinfection biosecurity devices. The controller may communicate instructions to the plurality of air disinfection biosecurity devices.

In some examples, each air disinfection biosecurity device also includes an antenna configured to communicate with one or more of the controller and one or more of the plurality of air disinfection biosecurity devices. The plurality of air disinfection biosecurity devices may include a first plurality of air disinfection biosecurity devices (first plurality) and a second plurality of air disinfection biosecurity devices (second plurality) wherein the controller communicates instructions directly to the first plurality and does not communicate instructions directly to the second plurality. In some examples, the first plurality may be configured to communicate the instructions to the second plurality, and one or more air disinfection biosecurity devices in the second plurality communicate the instructions to the other air disinfection biosecurity devices in the second plurality.

In some examples, the first plurality may include one or more air disinfection biosecurity devices, and the second plurality may include zero or more air disinfection biosecurity devices, and when the second plurality has zero air disinfection biosecurity devices the controller communicates directly with each air disinfection biosecurity device in the system. In some examples, the air disinfection biosecurity system may be a mesh network. The mesh network may include the controller and the plurality of air disinfection biosecurity devices that communicate with one or more of the controller and other air disinfection biosecurity devices in the mesh network.

According to another aspect of the present disclosure, a method of disinfecting a space with a defined volume may include delivering an electrical pulse from a high frequency module and sequential relay module set (HFM/SRM set) to a reaction chamber to generate cold plasma comprising hydrogen peroxide in the reaction chamber, directing fluid through the reaction chamber, and distributing the fluid and hydrogen peroxide into the space with a defined volume. The method may also include adjusting the intensity and frequency of the electrical pulse delivered from the HFM/SRM set, the adjustment being made based upon one or more of the size of the space with a defined volume, or the use and airflow of an HVAC system. In some examples, the method may include adjusting the frequency and intensity of the electrical pulse based on the size of the space with a defined volume or the cubic feet per minute of the HVAC or air handling system.

The method may further include directing the fluid and hydrogen peroxide through the HVAC components, the HVAC components including an HVAC duct, air handling system with cooling coils, and an air filter; and disinfecting and reducing scaling on the HVAC components via the hydrogen peroxide. In some examples, the method may include generating a predetermined amount of cold plasma in the reaction chamber. The method may also include providing a plurality of air disinfection biosecurity devices, providing the controller as a smart controller; and controlling via the smart controller the amount of hydrogen peroxide generated in the reaction chamber based on one or more variables, the variables including a high, a medium, or a low treatment output level, a time of day, and an arrangement of the plurality of air disinfection biosecurity devices relative to one another in the space.

These and other features of the present disclosure will become more apparent from the following description of the illustrative implementations.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 illustrates an exemplary embodiment of an Air Disinfection and Biosecurity (ADB) modulated dielectric barrier discharge (MDBD) system configured to eliminate airborne pathogens, virus, bacteria, mold and fungus in air circulated through an HVAC/air handling system;

FIG. 2 illustrates an exemplary embodiment of a standalone ADB MDBD system configured to eliminate airborne pathogens, virus, bacteria, mold, and fungus in air and on surfaces of a space with a defined volume without being connected to an HVAC/air handling system;

FIG. 3 illustrates an exemplary embodiment of an ADB MDBD system coupled to an HVAC/air handling system to treat air in a space with a defined volume.

FIG. 4 illustrates an exemplary embodiment of an ADB MDBD system coupled to an HVAC/air handling system; and

FIG. 5 illustrates an exemplary embodiment of a standalone ADB MDBD system coupled to a controller;

Corresponding reference numerals are used to indicate corresponding parts throughout the several views.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features, such as those representing devices, modules, instructions blocks and data elements, may be shown in specific arrangements and/or orderings for ease of description. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

In some embodiments, schematic elements used to represent blocks of a method may be manually performed by a user. In other embodiments, implementation of those schematic elements may be automated using any suitable form of machine-readable instruction, such as software or firmware applications, programs, functions, modules, routines, processes, procedures, plug-ins, applets, widgets, code fragments and/or others, for example, and each such instruction may be implemented using any suitable programming language, library, application programming interface (API), and/or other software development tools. For instance, in some embodiments, the schematic elements may be implemented using Java, C++, and/or other programming languages. Similarly, schematic elements used to represent data or information may be implemented using any suitable electronic arrangement or structure, such as a register, data store, table, record, array, index, hash, map, tree, list, graph, file (of any file type), folder, directory, database, and/or others, for example.

Further, in the drawings, where connecting elements, such as solid or dashed lines or arrows, are used to illustrate a connection, relationship, or association between or among two or more other schematic elements, the absence of any such connection elements is not meant to imply that no connection, relationship, or association can exist. In other words, some connections, relationships, or associations between elements may not be shown in the drawings so as not to obscure the disclosure. In addition, for ease of illustration, a single connecting element may be used to represent multiple connections, relationships, or associations between elements. For example, where a connecting element represents a communication of signals, data or instructions, it should be understood by those skilled in the art that such element may represent one or multiple signal paths (e.g., a bus), as may be needed, to effect the communication.

The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure.

Harmful pathogens and the spread of illness in the air and on surfaces pose an ongoing health issue for people. Prevention of cross-transmission and contamination is a challenge facing various facilities. Existing methods to address this challenge have included aggressive disinfection, cleaning, or treating with chemical sanitizers like bleach. These methods have limitations. They present a one-time treatment without continuous disinfection, and none of these solutions address airborne pathogens. Humans are also imperfect when it comes to cleaning and will often miss spots in a room.

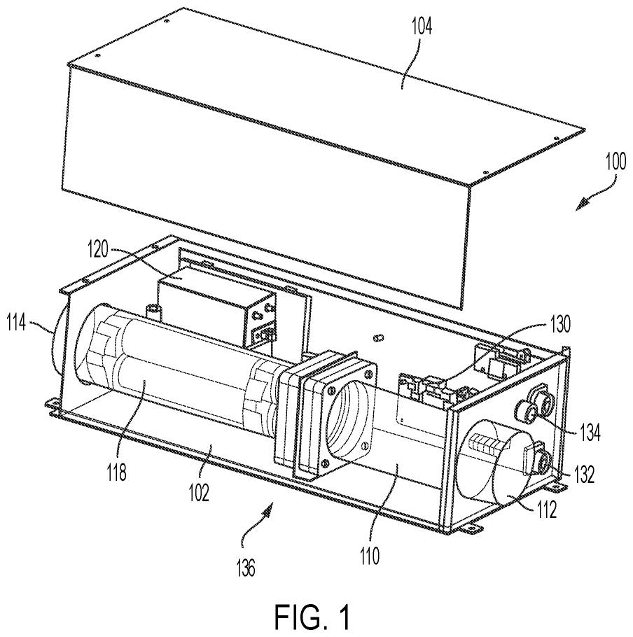

Air Disinfection and Biosecurity (ADB) devices and systems take in ambient air from a space with a defined volume and convert the humidity and oxygen in that air into plasma, wherein the plasma may include hydrogen peroxide, H₂O₂, a small amount of ozone (less than 5 parts per billion) and five other highly reactive molecules which is a natural pathogen elimination system. Gaseous hydrogen peroxide, when emitted in conjunction with the short-lived (micro-second to a second) highly reactive molecules (HRM), is effective for the safe and environmentally friendly inactivation of micro-organisms. Hydrogen peroxide naturally decomposes into water (H₂O) and oxygen (O₂). Utilizing ADB modulated dielectric barrier discharge (MDBD) 100 (see FIG. 1) specific and consistent cold plasma generation technology is safe for human occupants to rapidly eliminate pathogens.

ADB MDBD devices and systems 100 (hereinafter referred to as ADB MDBD systems for conciseness) provide safe, rapid, specific and consistent continual antimicrobial treatment, even in hard to reach areas. ADB MDBD systems 100 do not use dangerous chemicals, or hazardous irradiation, and provide long-term cost savings in chemical use and staff labor. ADB MDBD systems 100 do not require downtime and are safe to use in occupied spaces, when dangers from airborne pathogens, virus, bacteria, mold, and fungus in air and on surfaces may be prevalent. ADB MDBD systems 100 are chemical-free and residual-free, and do not require air filtration, ionizers, irradiation, or UV components. ADB MDBD systems 100 may provide safe, rapid, specific and consistent continual antimicrobial treatment with a fully automated system that utilizes no consumables.

ADB MDBD systems 100 use a non-thermal plasma technology that uses the ambient air as a vehicle to inactivate contaminating agents. ADB MDBD systems 100 create atmospheric cold (non-thermal) plasma. ADB MDBD technology is differentiated from other cold plasma technologies by its ability to generate highly consistent plasma capable of prolonged, self-sustaining propagation, making disinfecting agents available to neutralize harmful microbes in the air and on surfaces. The ADB MDBD 100 specific and consistent cold plasma generation is assured due to the use of high frequency modules (HFMs) paired with sequential relay modules (SRMs) (hereinafter referred to as HMF/SRM sets 120) each at exact settings to create a specific and consistent stable plasma and treatment. This propagation is continuously self-regulated for optimal performance and safety. The ADB MDBD systems 100 may each be engineered for their designated indoor environment application by analyzing one or more variables or characteristics of the HVAC system. For example, the ADB MDBD system 100 may be adjusted to create a precise amount of treatment based on one or more characteristics. One or more characteristics may include the volume of the space with a defined volume, the use of the space with a defined volume, and the cubic feet per minute (CFM) of the HVAC system that provides fluid, such as air, to the space with a defined volume. The ADB MDBD system 100 may be adjusted by adjusting the amount of voltage the control distribution board 120 delivers to the one or more HFM/SMR sets 120, thereby adjusting one or more of the frequency or pulse rate or intensity of the electrical pulse.

ADB MDBD systems 100 may generate a specific and consistent cold plasma from ambient air with several reactive sub-compounds of oxygen and peroxides including a particularly stable gas-phase hydrogen peroxide (well below regulatory emissions thresholds). These oxygen species collectively and actively eliminate airborne and surface-level microorganisms rapidly.

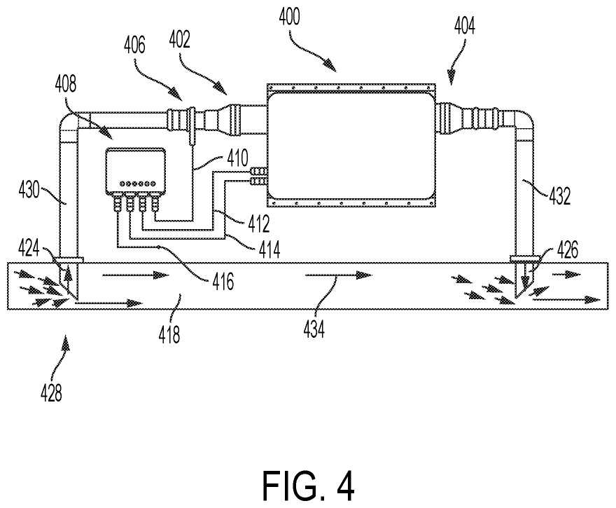

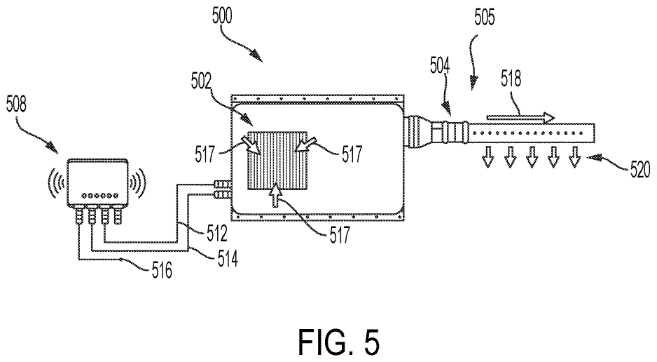

ADB MDBD systems 100 reinforce safety in common areas without impacting staffing or traffic flow. ADB MDBD systems 100 may include a controller 408, 508 (see FIGS. 4-5), such as an integrated smart controller or control system that controls the production quantities of the HRM. The ABD MDBD system 100 may include control boards and controls that may allow for the integration into a building management system. These integrated controls have fine-tuning capabilities to limit the amounts of production of the specific and consistent cold plasma components and cold plasma treatment with a dynamic environmental calibration for the main reactive species in this robust plasma generation. Cold plasma treatment levels can be controlled for high, medium, medium to low, or low treatment output level, time of day, grouping of multiple units or devices and other control options. The smart controller may ensure a monitored fully controlled and safe operation in compliance with regulatory safety guidelines.

ADB MDBD systems 100 may be integrated into existing HVAC or air handling systems or can be used as a standalone unit or device for individual rooms. ADB MDBD systems 100 are effective against airborne and surface viruses (including Coronavirus), as well as bacteria, mold, fungi, and allergens. Examples of the system effectiveness when used correctly are as follow:
airborne virus eliminated by 99.9999% in under 60 seconds;
surface virus eliminated by 99.9999% in under 15 minutes;
airborne Bacteria eliminated by 99.9999% in under 120 seconds;
surface bacteria eliminate 99.9999% in 30 minutes;
eliminates candida auris, escherichia coli, salmonella enteritis, human coronavirus 229E, and many more.

FIG. 1 illustrates an exemplary embodiment of an ADB MDBD system 100 configured to eliminate airborne pathogens, virus and bacteria in air circulated through an HVAC/air handling system. The ADB MDBD system may generate a treatment that includes delivering hydrogen peroxide and a small amount of ozone (less than 5 parts per billion) via air. In some examples, five other HRMs may also be generated by the ADB MDBD system 100, and the HRMs may last for around one second or less than one second. In these examples the HRMs may energize the effectiveness of the hydrogen peroxide. Hydrogen peroxide, a small amount of ozone (less than 5 parts per billion) in combination with the five HRMs may be an effective and natural pathogen elimination system, and may be referred to herein as the treatment. More specifically, the ADB MDBD system 100 may inject the treatment into air from the HVAC/air handling system, using the air from the HVAC/air handling system as a vehicle to deliver the treatment.

The ADB MDBD system 100 includes a housing 102, a cover 104, an airflow conduit 110, a reaction chamber 118, one or more HFM/SRM set 120, and a control distribution board 130. The housing 102 and cover 104 are configured to enclose the ADB MDBD system 100. FIG. 1 shows the cover 104 removed from the housing 102 so the internal components of the ADB MDBD system 100 may be seen.

The airflow conduit 110 extends through the housing 102 from an air inlet 112 to an air outlet 114, and the airflow conduit 110 encloses the reaction chamber 118. The airflow conduit 110 may be a single piece of conduit extending between the air inlet 112 and air outlet 114, or it may be a plurality of conduit pieces coupled together and extending between the air inlet 112 and air outlet 114. In the illustrative example of FIG. 1, the airflow conduit 110 may include two conduit pieces coupled together via a coupling 136. In some examples, the coupling 136 may have mesh extending at least partially into the airflow conduit 110, and the mesh may facilitate electrical pulses from the HFM/SMR set 120. In some examples, the air inlet 112 and air outlet 114 may be coupled to an HVAC duct 418 (see FIG. 4) and at least part of the air from the HVAC duct 418 may be directed to the ADB MDBD system 100 and then back into the HVAC airflow. Air flowing through the HVAC/air handling system may enter the ADB MDBD system 100 at the air inlet 112 and flow through the airflow conduit 110 towards the air outlet 114. As the air flows through the airflow conduit 110 it flows through the reaction chamber 118 where it may interact with ADB MDBD technology.

The reaction chamber 118 may be enclosed in the airflow conduit 110 and may be positioned between the air inlet 112 and air outlet 114. In some examples, the MDBD may comprise PVC with borosilicate glass tubing. A screen or mesh may be positioned on the inside and outside of the glass tubing.

The control distribution board 130 transmits and receives various signals used to control and coordinate operation of the ADB MDBD system 100. A data cable connection 132 may be used to connect the control distribution board 130 of the ADB MDBD system 100 to ventilation control systems for control and coordination of operation of the ADB MDBD system 100. In some systems, the control distribution board 130 may be incorporated into the ADB MDBD system 100. Alternatively, the control distribution board 130 may be wirelessly connected to ventilation control systems. The ADB MDBD system 100 may also include a process indicator 134 to indicate the operational status of the ADB MDBD system 100.

The ADB MDBD system 100 may also include one or more HFM/SRM sets 120. Each HFM/SRM set 120 includes an HFM and an SRM. The one or more HFM/SRM sets 120 produce specific modulated frequency signals that are directed at the reaction chamber 118 to generate hydrogen peroxide, among other things. The ADBD MDBD system may control the precise combination of the electric pulse by the HFM/SMR sets 120. This specific control creates a consistent plasma field that creates a consistent and stable output of hydrogen peroxide, a slight amount of ozone (O₃), and the five highly reactive molecules (HRMs), without producing any random harmful molecules or volatile organic compounds.

The ADB MDBD system 100 may control the precise combination of the electric pulse by the HFM/SMR sets 120 via the control distribution board 130. The control distribution board 130 may provide a voltage, or a range of voltages, to one or more HFM/SRM set 120. The voltage delivered to the one or more HFM/SRM set 120 may control the electric pulse rate and intensity output from the HFM/SRM set 120. Changing the output from the HFM/SRM set 120 via the control distribution board 130 may enable the ADB MDBD system 100 to create consistent and constant amounts of stable cold plasma. In some examples, increasing the voltage delivered to the HFM/SRM set 120 may increase the electric pulse rate and/or the intensity output from the HFM/SRM set 120. When the HFM/SRM set 120 electric pulse rate and/or intensity output increase, the amount of stable cold plasma treatment may increase. When the electric pulse rate and/or intensity output decrease, the amount of stable cold plasma treatment may decrease.

In some examples, the ADB MDBD system 100 may include a plurality of HFM/SRM sets 120, which may enable treatment of larger volume spaces with one ADB MDBD system 100. In some examples, one ADB MDBD system 100 may include 8 or more HFM/SRM sets 120. When the ADB MDBD system 100 has more than one HFM/SRM set 120, each HFM/SRM set 120 may operate dependently, or operate together wherein the output from one HFM/SRM set 120 may affect the output of the other HFM/SRM sets 120. In some examples, each HFM/SRM set 120 may operate independently from the other HFM/SRM sets 120 in the ADB MDBD system 100. In examples with a plurality of HFM/SRM sets 120, the HFM/SRM sets 120 may operate in parallel or in series.

In some examples, a plurality of ADB MDBD systems 100 may create a mesh network where the ADB MDBD systems 100 communicate with each other and/or a controller 408, 508. In some examples, one or more ADB MDBD systems 100 may include a communication board (not pictured) configured to communicate with the controller 408, 508 and/or the plurality of ADB MDBD systems 100 in the mesh network. In some examples, one or more of the plurality of ADB MDBD systems 100 in the mesh network may not include the communication board. The controller 408, 508 may control or adjust the settings of the ADB MDBD system 100. The settings may include the intensity and/or frequency of the electric pulse, and whether the ADB MDBD systems 100 are on or off, among other things.

In some embodiments, there may be two groups of ADB MDBD systems 100 and the controller 408, 508 may only directly communicate with the first group. In one such example, the controller 408, 508 may be out of range relative to the second group. In these embodiments, the controller 408, 508 may communicate instructions to one or more ADB MDBD systems 100 in the first group and one or more ADB MDBD systems 100 in the second group. However, the second group may not receive the communication because, for example, the second group may be out of range from the controller 408, 508. After the controller communicates the instructions to the first group, one or more ADB MDBD systems 100 in the first group may relay the instructions to one or more ADB MDBD systems 100 in the second group. In some examples, the ADB MDBD systems 100 in the second group may communicate with other ADB MDBD systems 100 in the second group, until either (i) the instruction reaches each ADB MDBD systems 100 in the first and second groups, or (ii) the instructions reach each of the intended ADB MDBD systems 100. In some examples, one or more ADB MDBD systems 100 may have an antenna which may greatly extend the communication range of the one or more ADBD MDBD systems 100.

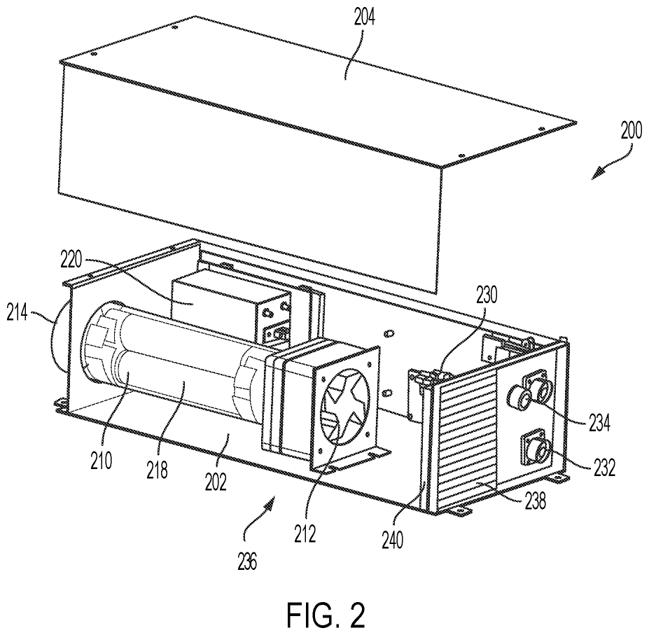

FIG. 2 illustrates an exemplary embodiment of a standalone ADB MDBD system 200 configured to eliminate airborne pathogens, virus, and bacteria in air of a space with a defined volume. The ADB MDBD system 200 may eliminate airborne pathogens, virus, and bacteria in air of a space with a defined volume without being connected to an HVAC/air handling system. In some examples the ADB MDBD system 200 is portable. The ADB MDBD system 200 includes a housing 202, a cover 204, an airflow conduit 210, a reaction chamber 218, a fan 212, HFM/SRM set(s) 220, and a control distribution board 230. The housing 202 and cover 204 are configured to enclose the ADB system 200. The ADB MDBD system 200 may include a vent 238 positioned in the housing 202, as illustrated in FIG. 2. In some examples, the vent 238 may be a sidewall vent 238, or the vent may be in the cover 204. FIG. 2 shows the cover 204 removed from the housing 202 so the internal components of the ADB system 200 may be seen.

The airflow conduit 210 extends through the housing 202 from the fan 212 to an air outlet 214, and the airflow conduit 210 encloses the reaction chamber 218. The fan 212 draws air from outside the airflow conduit 210, such as air from a space with a defined volume, or a room, and delivers the air into the airflow conduit 210 of the ADB MDBD system 200, delivering the air through the airflow conduit 210 and to the air outlet 214. In some examples, the ADB MDBD system may include a filter 240 coupled to the vent 238. The filter 240 may be positioned between the fan 212 and the vent 238 to filter dust, particles, and other debris. In some examples, the filter 240 may be a sidewall filter coupled to the vent 238. The fan 212 may draw air through the vent 238, and move air through the filter 240 and into the airflow conduit 210. As the air flows through the airflow conduit 210 it flows through the reaction chamber 218 where the air is treated by ADB MDBD technology.

In some examples the fan speed may be adjustable. In the illustrative example of FIG. 2, the airflow conduit 210 may include one piece of conduit that may be coupled to the fan 212 via a coupling 236. In some examples, the coupling 236 includes mesh extending at least partially into the airflow conduit 210, and the mesh may facilitate electrical pulses from the HFM/SMR set 220.

The reaction chamber 218 may be enclosed in the airflow conduit 210 and may be positioned between the fan 212 and air outlet 214. In some examples, the MDBD may comprise PVC with borosilicate glass tubing. A screen or mesh may be positioned on the inside and outside of the glass tubing.

The control distribution board 230 transmits and receives various signals used to control and coordinate operation of the ADB MDBD system 200. A data cable connection 232 can be used to connect the control distribution board 230 of the ADB system 200 to other systems for control and coordination of operation of the ADB system 200. Alternatively, the control distribution board 230 can be wirelessly connected to the other systems. The ADB MDBD system 200 can also include a process indicator 234 to indicate the operational status of the ADB MDBD system 200. The ADB system 200 may also include one or more HFM/SRM sets 220 configured to produce specific modulated frequency signals used in treating the air that flows through the reaction chamber 218.

In some examples, the ADB MDBD system 200 may include a plurality of HFM/SRM sets 220, which may enable treatment of larger volume spaces with one ADB MDBD system 200. In some examples, one ADB MDBD system 200 may include 8 or more HFM/SRM sets 220. When the ADB MDBD system 200 has more than one HFM/SRM set 220, each HFM/SRM set 220 may operate together, wherein the output from one HFM/SRM set 220 may affect the output of the other HFM/SRM sets 220. In some examples, each HFM/SRM set 220 may operate independently from the other HFM/SRM sets 220 in the ADB MDBD system 200.

In some examples, a plurality of ADB MDBD systems 200 may create a mesh network. The mesh network may comprise a controller 408, 508 and a plurality of ADBD MDBD systems 200 that communicate with each other and/or the controller 408, 508. In some embodiments, there may be two groups of ADB MDBD systems 200 and the controller 408, 508 may only directly communicate with the first group. In one such example, the controller 408, 508 may be out of range relative to the second group. In these embodiments, the controller 408, 508 may communicate instructions to one or more ADB MDBD systems 200 in the first group and one or more ADB MDBD systems 200 in the second group. However, the second group may not receive the communication because, for example, the second group may be out of range from the controller 408, 508. After the controller 408, 508 communicates the instructions to the first group, one or more ADB MDBD systems 200 in the first group may relay the instruction to one or more ADB MDBD systems 200 in the second group. In some examples, the ADB MDBD systems 200 in the second group may communicate with other ADB MDBD systems 200 in the second group, until either (i) the instruction reaches each ADB MDBD systems 200 in the first and second groups, or (ii) the instructions reach each of the intended ADB MDBD systems 200. In some examples, one or more ADB MDBD system 200 may have an antenna.

In some examples, the mesh network may include a combination of ADB MDBD systems 100, 200, wherein the systems 100, 200 includes one or more ADB MDBD systems 100 that are coupled to the HVAC system, and one or more ADB MDBD systems 200 that are standalone units or devices. In these examples, both ADB MDBD systems 100, 200 may be controlled by one controller 400, 500, or may be controlled by more than one controller 400, 500.

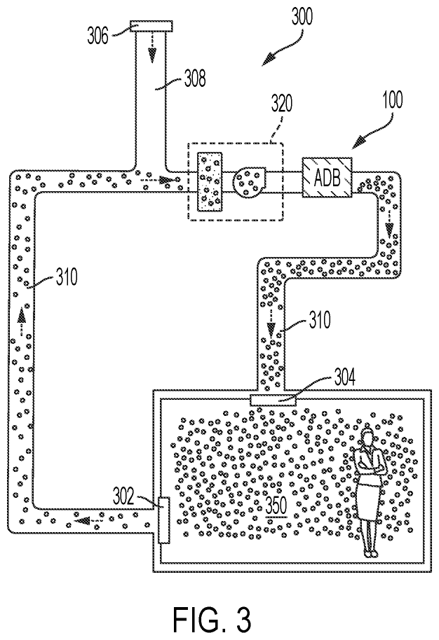

The ADB MDBD systems 100, 200 draw ambient air from a space with a defined volume, or a room, through the reaction chamber 118, 218 where it is treated. In some examples, as illustrated in FIG. 3, the ADB MDBD system 100, 200 may also draw outside air. The ADB systems MDBD 100, 200 may also include a smart controller for ease of use. ADB MDBD technology may inactivate 90 percent of airborne viruses (including SARS-COV-2) in about 16 seconds, and may achieve 99.9 percent inactivation in about 30 seconds. ADB MDBD technology has been found to reduce airborne viruses by 99.9999 percent, virtually eliminating any potential for transmission, in about 60 seconds. ADB MDBD technology may also eliminate 99.9 percent of viruses on surfaces (steel, plastic, fabric, linoleum), eliminate any potential for transmission, in about 11-12 minutes. ADB MDBD technology may eradicate 99.9999 percent of airborne bacteria in under 120 seconds, and may eliminate 99.9 percent of surface bacteria (steel, plastic, fabric, linoleum) in about 11-13 minutes. ADB MDBD technology has also been found to be effective in neutralizing molds, fungus, mildews and some volatile organic compounds (VOCs). Additionally, the individual ADB MDBD systems 100, 200 include a communication feature that allow each to communicate not only with the controller but with each other to form a mesh network amplifying all control signals.

Hydrogen peroxide ions are a natural pathogen elimination system. The ADB MDBD systems 100, 200 take in ambient air from the space with a defined volume, or the room, and convert the humidity and oxygen in the air into hydrogen peroxide, which has a half-life of 10 to 20 minutes. Gaseous hydrogen peroxide is effective for the safe and environmentally friendly inactivation of micro-organisms. Unlike other low temperature sterilization technologies, ADB MDBD systems 100, 200 create a consistent plasma field that creates a consistent and stable output of hydrogen peroxide, slight amount of ozone (O₃), the five highly reactive molecules (HRMs), without producing any random harmful molecules. The hydrogen peroxide gaseous treatment along with the HRMs naturally decomposes into water (H₂O) and oxygen (O₂).

The ADB MDBD systems 100, 200 are both safe for human occupants, and effective in the rapid elimination of pathogens. Hydrogen peroxide has been used by the medical community for over 150 years. Hydrogen peroxide is used in toothpaste, mouthwash, and household cleaners. The EPA has recognized hydrogen peroxide ions as an effective infection control and microbial treatment technology. In addition to inactivating viruses and killing bacteria, the specific and consistent cold plasma treatment will kill preexisting mold, fungus, mold spores, etc., and bring the area to a very low infestation rate. The specific and consistent cold plasma treatment will also reduce or eliminate organic based odors.

The ADB MDBD systems 100, 200 may provide safe, rapid, and continual antimicrobial treatment, even in hard-to-reach areas because air delivers the treatment, and the treatment may be delivered wherever air is delivered. The ADB MDBD systems 100, 200 do not use dangerous chemicals, or hazardous irradiation. The ADB MDBD systems 100, 200 provide long-term cost savings by minimizing and/or eliminating chemical use and/or staff labor. The ADB MDBD systems 100, 200 do not require downtime and are safe to use in occupied spaces. The ADB MDBD systems 100, 200 are chemical-free and residual-free. The ADB MDBD systems 100, 200 also do not require air filtration, ionizers, irradiation, or harmful UV components.

FIG. 3 illustrates an exemplary embodiment of the ADB MDBD system 100 coupled to an HVAC system 300 to treat the air in an occupied space with a defined volume 350. The HVAC system 300 is connected to the space with a defined volume 350 by one or more air circulation ducts 310 that are connected to one or more return vents 302 and one or more supply vents 304. The HVAC system 300 may draw air from the space with a defined volume 350 into the air circulation ducts 310 through one of the return vents 302. The HVAC system 300 may also draw outside air into the air circulation ducts 310 through an outside vent 306 and an optional outside air duct 308. A mixture of air from the space with a defined volume 350 (through the return vents 302) and outside air (through the outside vent 306) may be drawn into an air handling unit 320 where the HVAC system 300 conditions the air. After passing through the air handling unit 320, a portion of the air may be routed to the ADB MDBD system 100 for disinfection and treatment. The ADB MDBD system 100 can provide a specific and consistent cold plasma treatment for disinfection and treatment of the incoming air from the air handling unit 320. After passing through the ADB MDBD system 100, the treated air may be routed through the air circulation ducts 310 back to the space with a defined volume 350 through the supply vents 304.

The air and hydrogen peroxide treatment from the ADB MDBD system 100 may provide continuous sanitization of the space with a defined volume 350. The air and hydrogen peroxide treatment from the ADB MDBD system 100 may also provide continuous sanitization of the air circulation ducts 310 and the air handling unit 320 of the HVAC system 300. The air and hydrogen peroxide may also disinfect HVAC components. The HVAC components may include an HVAC duct, the air handling system with cooling coils, and an air filter. In addition to disinfecting the space with a defined volume and the HVAC components, the hydrogen peroxide may prevent residue buildup, such as scaling on the cooling coils.

FIG. 4 illustrates an exemplary embodiment of an ADB MDBD system 400 coupled to an HVAC/air handling system 428, the ADB MDBD system 400 configured to eliminate airborne pathogens, virus, bacteria, mold and fungus in air circulated through the HVAC/air handling system 428. The ADB MDBD system 400 may be similar to the ADB MDBD system 100 of FIG. 1. FIG. 4 shows the cover attached to the housing, enclosing the internal components of the ADB MDBD system 400. Although not shown in FIG. 4, the ABD MDBD system 400 may include the airflow conduit 110, reaction chamber 118, one or more HFM/SRM sets 120, and control distribution board 130, as illustrated in FIG. 1.

The ADB MDBD system 400 may be coupled to the HVAC/air handling system 428 via piping 430, 432. The ADB MDBD system 400 may be positioned between an air inlet 402 and an air outlet 404, wherein the air inlet 402 is coupled to the inlet piping 430 and the air outlet 404 is coupled to the outlet piping 432. Air may be directed through an HVAC duct 418 in the direction of airflow 434, thereby bypassing the ADB MDBD system. Additionally, a portion of the air from the HVAC/air handling system 428 may be directed to the ADB MDBD system 400 via inlet and outlet piping 430, 432. More specifically, inlet piping 430 may route a portion of the air from the HVAC/air handling system 428 to the ADB MDBD system 400, and outlet piping 432 may route air and/or treatment from the ADB MDBD system 400 to the HVAC/air handling system 428.

In some examples, inlet piping 430 may include, or be coupled to, an entrance 424 that is positioned proximal to the air inlet 402. The outlet piping 432 may include, or be coupled to, an exit 426 that may be positioned distal to the air outlet 404. The entrance 424 may be angled relative to the direction of the airflow 434, and the exit 426 may be angled relative to the direction of the airflow 434. In some examples, the angle between the entrance 424 and the airflow may be between 35-90 degrees, and the angle between the exit 426 and the airflow 434 may be between 35-90 degrees. In some examples, each angle may be approximately 45 degrees. The entrance 424 may be configured to a portion of the air from the HVAC/air handling system 428 to the ADB MDBD system 400, and the exit 426 may be configured to direct air and/or treatment from the ADB MDBD system 400 back into the HVAC/air handling system 428.

The inlet piping 430 may include a mass airflow (MAF) sensor 406 configured to detect the mass flow rate of the air. In some examples, the MAF sensor 406 may be positioned proximal to the inlet 402, or between entrance 424 and inlet 402, and air may pass the MAF sensor 406 before being directed to inlet 402.

The ABD MDBD system 400 may be coupled to a controller 408. The controller 408 may communicate with one or more ABD MDBD systems 400 and may control, among other things, the intensity and/or frequency of voltage sent from the control distribution board 130 to one or more HFM/SRM sets 120 to control the electric pulse rate and/or intensity of the HFM/SRM sets 120 (see FIG. 1). In some examples, the controller 408 may turn the HFM/SRM sets 120 on and off. The controller 408 may be a drive controller, or a DRV controller.

The controller 408 may include an MAF signal cable 410, a data cable 412, a control cable 414, and a power cable 416. The MAF sensor 406 may communicate with the controller 408 via the MAF signal cable 410. In some examples, the MAF sensor 406 may wirelessly communicate with the controller 408 The MAF sensor 406 may communicate the mass flow rate to the controller 408 via the MAF signal cable. The ADB MDBD system 400 may include a switch and the controller 408 may actuate the switch to turn the ADB MDBD system 400 on and off. In some examples, the MAF sensor 406 may be the switch that turns the ADB MDBD system 400 on and off. In other examples, a separate switch may turn the ADB MDBD system 400 on and off and the MAF sensor 406 may actuate the switch via the controller 408 when the MAF sensor 406 detects a change in airflow. In one example, the ADB MDBD system 400 may be off and the MAF sensor 406 may not detect airflow. In this example, when the MAF sensor 406 detects airflow, the MAF sensor 406 may communicate with the controller 408 and the controller 408 may actuate the switch and turn the ADB MDBD system 400 on. In a second example, the ADB MDBD system 400 may be on and the MAF sensor 406 may detect airflow. In this second example, when the MAF sensor 406 does not detect airflow, the MAF sensor 406 may communicate with the controller 408 and the controller 408 may actuate the switch to turn off the ADB MDBD system 400. In some examples, there may be a time delay between when the MAF sensor 406 detects a change in airflow and when the MAF sensor 406 and/or switch is activated.

The ADB MDBD system 400 may communicate with the controller 408 via the data cable 412 and/or the control cable 414. In some examples, the controller 408 may provide instructions to the ADB MDBD system 400 via the control cable 414. In some examples, the instructions may include a particular voltage and/or frequency of the voltage to be sent to the one or more HFM/SRM sets 120. The controller 408 may be powered by a power cable 416, however the controller 408 may also be battery powered. In some examples, the controller 408 may wirelessly communicate with the MAF sensor 406 and/or the ABD MDBD system 400.

FIG. 5 illustrates an exemplary embodiment of a standalone ADB MDBD system 500 configured to eliminate airborne pathogens, virus, and bacteria in air of a space with a defined volume, or a room. The ADB MDBD system 500 may be similar to the ADB MDBD system 200 of FIG. 2, and may eliminate airborne pathogens, virus, and bacteria in air of a space with a defined volume, or a room, without being connected to an HVAC/air handling system. FIG. 5 shows the ADB MDBD system 500 with the cover coupled to the housing enclosing the internal parts. Although not shown in FIG. 5, the ADB MDBD system 500 includes the airflow conduit 210, fan 212, reaction chamber 218, one or more HFM/SRM set, and control distribution board 230, as illustrated in FIG. 2.

The ADB MDBD system 500 may include an inlet 502 configured to route air to the ADB MDBD system 500 and an outlet 504 configured to route air and/or treatment from the ADB MDBD system 500. In some examples, the inlet 502 may be a vent, and the vent may allow the fan 212 (see FIG. 2) to draw air from outside the ADB MDBD system 500. The outlet 504 may be coupled to a distribution pipe 505 which distributes air and/or treatment. In some examples, air and/or treatment may be directed at least partially through the air distribution pipe 505 in a first direction 518, and air may be distributed from the air distribution pipe 505 in a second direction 520. While exemplary illustration of FIG. 5 shows air being distributed in a particular direction (e.g., in direction 520), it should be understood that air may be distributed from the distribution pipe 505 in any direction.

In some examples, the fan 212 may draw air 517 into the housing 102 of the ABD MDBD system 500 and the fan 212 may direct air through the airflow conduit 210 and past the reaction chamber 218. In these examples, air and/or treatment may be routed from the ADB MDBD system 500 via the outlet 504, and the air and/or treatment may be directed at least partially through the distribution pipe 505. Air and/or treatment may move through the air distribution pipe 505 in the first direction 518 and may be distributed from the air distribution pipe 505 in the second direction 520.

The ABD MDBD system 500 may be coupled to a controller 508. The controller 508 may communicate with one or more ABD MDBD system 500 and may control, among other things, the intensity and/or frequency of voltage sent from the control distribution board 230 to one or more HFM/SRM sets 220 to control the pulse rate and/or intensity of the HFM/SRM sets 220. In some examples, the controller 508 may turn one or more HFM/SRM set on and off. The controller 508 may be a drive controller or a DRV controller, and may wirelessly communicate to the ABD MDBD system 500 and/or communicate with the ABD MDBD system 500 via one or more wired connection.

The controller 508 may include a data cable 512, a control cable 514, and a power cable 516. The ADB MDBD system 500 may communicate with the controller 508 via the data cable 512 and/or the control cable 514. In some examples, the controller 508 may provide instructions to the ADB MDBD system 500 via the control cable 514, and the instructions may include a particular voltage and/or frequency of the voltage to be sent to the one or more HFM/SRM sets 220. The controller may be powered by a power cable 416 or may be battery powered.

While the disclosure has been illustrated and described in detail in the drawings and foregoing description, such illustration and description is to be considered as exemplary and not restrictive in character, it being understood that illustrative embodiment(s) have been shown and described and that all changes and modifications that come within the spirit of the disclosure are desired to be protected. It will be noted that alternative embodiments of the present disclosure may not include all of the features described yet still benefit from at least some of the advantages of such features. Those of ordinary skill in the art may readily devise their own implementations that incorporate one or more of the features of the present disclosure and fall within the spirit and scope of the present invention as defined by the appended claims.

1. A fluid disinfection biosecurity device comprising:
a housing with an inlet and an outlet;
a conduit positioned between the inlet and the outlet, the conduit configured to direct fluid from the inlet to the outlet;
a reaction chamber positioned in the conduit between the inlet and the outlet;
a high frequency module and sequential relay module set (HFM/SRM set) configured to transmit an electrical pulse at a frequency and an intensity, the HFM/SRM set positioned within the housing and configured to transmit the electrical pulse to the reaction chamber; and
a distribution board coupled to the HFM/SRM set, the distribution board configured to control the frequency and intensity of the electrical pulse by providing a voltage to the HFM/SRM set;
wherein the electrical pulse from the HFM/SRM set is transmitted to the reaction chamber at a predefined intensity and frequency;
wherein the reaction chamber is configured to produce hydrogen peroxide via the interaction with the electrical pulse from the HFM/SRM set, and the hydrogen peroxide being mixed with the fluid in the conduit and being transferred out of the housing through the outlet.

2. The fluid disinfection biosecurity device of claim 1, wherein the housing is coupled to one or more of an HVAC or air handling system.

3. The fluid disinfection biosecurity device of claim 1, further comprising a plurality of HFM/SRM sets positioned in the housing, wherein each HFM/SRM set of the plurality of HFM/SRM sets operates independently of the other plurality of HFM SRM sets.

4. The fluid disinfection biosecurity device of claim 1, wherein the reaction chamber is configured to receive the electrical pulse from the HFM/SRM set and produce a predetermined amount of gaseous hydrogen peroxide and ozone, wherein the gaseous hydrogen peroxide and ozone is produced free of any harmful or volatile organic compounds.

5. The fluid disinfection biosecurity device of claim 1, further comprising a controller coupled to the distribution board, the controller configured to control the intensity or frequency of the electric pulse.

6. The fluid disinfection biosecurity device of claim 5, further comprising a mass airflow sensor coupled to the controller, the mass airflow sensor positioned proximal to the inlet and configured to detect the mass flow rate of the fluid.

7. The fluid disinfection biosecurity device of claim 5, further comprising a fan coupled to the conduit, the fan being controllable by the controller and configured to move the fluid into the conduit and deliver the mixture of hydrogen peroxide and fluid to a space with a defined volume.

8. The fluid disinfection biosecurity device of claim 7, wherein the fluid disinfection biosecurity device is a standalone device separate from an HVAC or air handling system.

9. The fluid disinfection biosecurity device of claim 1, further comprising a plurality of HFM/SRM sets, wherein the plurality of HFM/SRM sets are positioned in series or parallel with respect to one another within the housing.

10. An air disinfection biosecurity system comprising:
a plurality of air disinfection biosecurity devices, each air disinfection biosecurity device comprising:
a housing with an inlet and an outlet;
a reaction chamber positioned within the housing between the inlet and outlet;
a conduit configured to move fluid from the inlet through the reaction chamber to the outlet;
a high frequency module and sequential relay module set (HFM/SRM set) configured to deliver an electrical pulse to the reaction chamber to generate hydrogen peroxide, the electrical pulse having a predetermined intensity and frequency;
a distribution board coupled to the HFM/SRM set configured to control the electrical pulse; and
a controller communicatively coupled to one or more of the plurality of air disinfection biosecurity devices;
wherein the controller communicates instructions to the plurality of air disinfection biosecurity devices.

11. The air disinfection biosecurity system of claim 10, wherein each air disinfection biosecurity device further comprises an antenna configured to communicate with one or more of the controller and one or more of the plurality of air disinfection biosecurity devices.

12. The air disinfection biosecurity system of claim 10, wherein the plurality of air disinfection biosecurity devices comprises a first plurality of air disinfection biosecurity devices and a second plurality of air disinfection biosecurity devices wherein the controller communicates instructions directly to the first plurality of air disinfection biosecurity devices and does not communicate instructions directly to the second plurality of air disinfection biosecurity devices;
wherein the first plurality of air disinfection biosecurity devices is configured to communicate the instructions to the second plurality of air disinfection biosecurity devices, and one or more air disinfection biosecurity devices in the second plurality of air disinfection biosecurity devices communicate the instructions to the other air disinfection biosecurity devices in the second plurality of air disinfection biosecurity devices.

13. The air disinfection biosecurity system of claim 12, wherein the first plurality of air disinfection biosecurity devices includes one or more air disinfection biosecurity devices, and the second plurality of air disinfection biosecurity devices includes zero or more air disinfection biosecurity devices, and when the second plurality of air disinfection biosecurity devices has zero air disinfection biosecurity devices the controller communicates directly with each air disinfection biosecurity device in the system.

14. The air disinfection biosecurity system of claim 10, wherein the air disinfection biosecurity system is a mesh network, the mesh network comprising the controller and the plurality of air disinfection biosecurity devices that communicate with one or more of the controller and other air disinfection biosecurity devices in the mesh network.

15. A method of disinfecting a space with a defined volume, the method comprising:
delivering an electrical pulse from a high frequency module and sequential relay module set (HFM/SRM set) to a reaction chamber to generate cold plasma comprising hydrogen peroxide in the reaction chamber;
directing fluid through the reaction chamber; and
distributing the fluid and hydrogen peroxide into the space with a defined volume.

16. The method of claim 15, further comprising adjusting the intensity and frequency of the electrical pulse delivered from the HFM/SRM set, the adjustment being made based upon one or more of the size of the space with a defined volume, or the use and airflow of an HVAC system.

17. The method of claim 16, further comprising adjusting the frequency and intensity of the electrical pulse based on the size of the space with a defined volume or the cubic feet per minute of the HVAC or air handling system.

18. The method of claim 15, further comprising:
directing the fluid and hydrogen peroxide through the HVAC components, the HVAC components including an HVAC duct, air handling system with cooling coils, and an air filter; and disinfecting and reducing scaling on the HVAC components via the hydrogen peroxide.

19. The method of claim 15, further comprising generating a predetermined amount of cold plasma in the reaction chamber.

20. The method of claim 15, further comprising:
providing a plurality of air disinfection biosecurity devices;
providing the controller as a smart controller; and
controlling via the smart controller the amount of hydrogen peroxide generated in the reaction chamber based on one or more variables, the variables including a high, a medium, or a low treatment output level, a time of day, and an arrangement of the plurality of air disinfection biosecurity devices relative to one another in the space.
